(19) United States
(12) Patent Application Publication
(10) Pub. No.: US 2012/0052010 A1
(43) Pub. Date: Mar. 1, 2012

(54) IMAGING TUMOR PERFUSION, OXIDATIVE METABOLISM USING DYNAMIC ACE PET IN PATIENTS WITH HEAD AND NECK CANCER DURING RADIOTHERAPY

(75) Inventors: Jens Sorensen, Uppsala (SE); Silvia Johansson, Umea (SE); Aijun Sun, Umea (SE)

(73) Assignee: GE HEALTHCARE LIMITED, Little Chalfont (GB)

(21) Appl. No.: 13/266,824

(22) PCT Filed: Apr. 29, 2010
(86) PCT No.: PCT/US2010/032870

§ 371 Date: Oct. 28, 2011

Related U.S. Application Data
(60) Provisional application No. 61/174,008, filed on Apr. 30, 2009.

(51) Int. Cl. A61K 51/04; C07C 53/18; A61N 5/00; C07C 53/10

(52) U.S. Cl. 424/1.89; 424/1.81; 562/607; 562/605; 600/1

(57) ABSTRACT

The present invention provides methods of using optimal PET tracers for diagnosing head and neck cancer. Non-invasive methods for assessing tumor perfusion and oxidative metabolism for in vivo imaging with PET tracers that are suitable for uses in radiation therapy (RT) in head and neck cancer and evaluation of salivary gland function are provided. A pharmaceutical comprising the PET tracer and a kit for the preparation of the pharmaceutical are provided as well.

### CROSS-REFERENCE TO RELATED APPLICATIONS

This application is a filing under 35 U.S.C. §371 and claims priority to international patent application number PCT/US2010/032870 filed Apr. 29, 2010, published on Nov. 4, 2010 as WO 2010/127054, which claims priority to U.S. provisional patent application No. 61/174,008 filed on Apr. 30, 2009.

### FIELD OF THE INVENTION

The present invention relates to the development of Positron Emission Tomography (PET) tracers that could be used for imaging for radiotherapy in head and neck cancer. The present invention specifically relates to non-invasive methods for assessing tumor perfusion and oxidative metabolism for in vivo imaging uses of PET tracers that are suitable in radiation therapy (RT) in head and neck cancer and in evaluating salivary gland function. A pharmaceutical comprising the compound and a kit for the preparation of the pharmaceutical are also provided.

### BACKGROUND OF THE INVENTION

Tracers labeled with short-lived positron emitting radionuclides (e.g. ¹⁸F and ¹¹C) are the positron-emitting nuclide of choice for many receptor imaging studies. Accordingly, radiolabeled ligands have great clinical potential because of their utility in Positron Emission Tomography (PET) to quantitatively detect and characterize a wide variety of diseases.

Head and neck squamous cell carcinoma is curable when diagnosed at an early stage. Both accurate diagnosis and staging of the tumors are important for prognosis and determination of treatment strategies. Conventional anatomic imaging techniques, such as computed tomography (CT), magnetic resonance imaging (MRI) and ultrasonography, are routinely used for evaluation of size and local tumor extend. However, there are inherent limitations associated with all these techniques (Vermeersch H, Loose D, Ham H, Otte A, Van de Wiele C. Nuclear medicine imaging for the assessment of primary and recurrent head and neck carcinoma using routinely available tracers. Eur J Nucl Med Mol Imaging 2003; 30:1689-700).

Positron emission tomography (PET) may improve the ability to noninvasively detect the biological characteristics of the tumors. ¹⁸F-fluoro-2-deoxy-D-glucose (FDG) PET has been widely applied for staging of the tumor, distinguishing tumor recurrence and predicting treatment response in head and neck cancer (Greven K M. Positron-emission tomography for head and neck cancer. Semin Radiat Oncol 2004; 14:121-9, Schwartz D L, Ford E C, Rajendran J, Yueh B, Coltrera M D, Virgin J, et al. FDG-PET/CT-guided intensity modulated head and neck radiotherapy: a pilot investigation. Head Neck 2005; 27:478-87, Avril N E, Weber W A. Monitoring response to treatment in patients utilizing PET. Radiol Clin North Am 2005; 43:189-204). PET is also increasing its use in delineation of gross tumor volume (Paulino A C, Johnstone P A. FDG-PET in radiotherapy treatment planning: Pandora's box? Int J Radiat Oncol Biol Phys 2004; 59:4-5).

FDG is an analog of glucose with high uptake in malignant cells, due to increased energy requirement (Strauss L G, Conti P S. The applications of PET in clinical oncology. J Nucl Med 1991; 32:623-48). However, FDG is not a specific tumor marker. It accumulates in inflammatory tissues and it also has limitations in finding well differentiated tumors (Goerres G W, Von Schulthess G K, Hany T F. Positron emission tomography and PET CT of the head and neck: FDG uptake in normal anatomy, in benign lesions, and in changes resulting from treatment. AJR Am J Roentgenol 2002; 179:1337-43, Delbeke D, Coleman R E, Guiberteau M J, Brown M L, Royal H D, Siegel B A, et al. Procedure guideline for tumor imaging with 18F-FDG PET/CT 1.0. J Nucl Med 2006; 47:885-95). Development of new tracers for improving the efficiency of PET imaging in head and neck cancer is therefore warranted.

Several recent studies have demonstrated that ¹¹C-acetate (“ACE”) might be a useful tracer for a few cancer types, such as lung cancer, hepatocellular carcinoma, renal cancer, prostate cancer and astrocytomas (Higashi K, Ueda Y, Matsunari I, Kodama Y, Ikeda R, Miura K, et al. 11C-acetate PET imaging of lung cancer: comparison with 18F-FDG PET and 99 mTc-MIBI SPET. Eur J Nucl Med Mol Imaging 2004; 31:13-21, Ho C L, Yu S C, Yeung D W. 11C-acetate PET imaging in hepatocellular carcinoma and other liver masses. J Nucl Med 2003; 44:213-21, Fricke E, Machtens S, Hofmann M, van den Hoff J, Bergh S, Brunkhorst T, et al. Positron emission tomography with 11C-acetate and 18F-FDG in prostate cancer patients. Eur J Nucl Med Mol Imaging 2003; 30:607-11, Shreve P, Chiao P C, Humes H D, Schwaiger M, Gross M D. Carbon-11-acetate PET imaging in renal disease. J Nucl Med 1995; 36:1595-601, Liu R S, Chang C P, Chu L S, Chu Y K, Hsieh H J, Chang C W, et al. PET imaging of brain astrocytoma with 1-(11)C-acetate. Eur J Nucl Med Mol Imaging 2006; 33:420-7). Ho et al (Ho C L, Yu S C, Yeung D W. 11C-acetate PET imaging in hepatocellular carcinoma and other liver masses. J Nucl Med 2003; 44:213-21) reported that well-differentiated hepatocellular carcinoma displayed increased ACE uptake and minimal FDG uptake. These findings indicated that ACE and ¹⁸F-acetate may have a high sensitivity and specificity as a radiotracer complementary to FDG in the PET imaging of hepatocellular carcinoma.

The present knowledge of ACE-PET and ¹⁸F-acetate-PET in head and neck cancer is, however, sparse. Head and neck cancer is a lethal malignancy for which combinations of surgery, chemotherapy and/or radiation therapy (RT) are used for curative intent. Therefore, there is a growing need for developing new molecular imaging technologies with high sensitivity and specificity in this field. A growing body of evidence links alterations of the intermediary metabolism in cancer to treatment outcome. Accordingly, the present invention presents a non-invasive method in vivo for assessment of tumor perfusion and oxidative metabolism in a subject using ACE-PET. This method can be used to document a metabolic abnormality, predictive of a poor response to radiotherapy. Thus restoration of tumor oxidative metabolism is a potential target for improvement in cancer therapy.

Discussion or citation of a reference herein shall not be construed as an admission that such reference is prior art to the present invention.

### SUMMARY OF THE INVENTION

In view of the long felt need for optimal staging of head and neck cancer, more advanced non-invasive methods for assessment of tumor perfusion and oxidative metabolism are needed. These methods would comprise of administrating a PET tracer in a subject with head and neck cancer. A pharmaceutical comprising the compound and a kit for the preparation of the pharmaceutical are also provided.

In one embodiment of the invention, a non-invasive method for assessment of tumor perfusion and oxidative metabolism, comprising in vivo administration of a PET tracer in a subject with head and neck cancer is disclosed wherein the PET tracer may be ACE or ¹⁸F-acetate.

Another embodiment of the present invention is a non-invasive method for assessment of tumor perfusion and oxidative metabolism in a subject with head and neck cancer, comprising administration of a pharmaceutical composition of a PET tracer. Still a further embodiment of the current invention discloses a pharmaceutical composition comprising a PET tracer, together with a biocompatible carrier in a form suitable for mammalian administration.

Yet in another embodiment of the invention, a non-invasive method for assessment of tumor perfusion and oxidative metabolism comprising a personalized RT treatment for head and neck cancer in a subject comprising administering a pharmaceutical composition of a compound of a PET tracer, tracing tumor delineation and giving personalized radiation dose amount in the tumor is disclosed.

The present invention also provides a non-invasive method for assessment of tumor perfusion and oxidative metabolism comprising a personalized RT treatment for head and neck cancer in a subject comprising administering a pharmaceutical composition of a compound of a PET tracer, evaluating salivary gland function, and giving personalized radiation dose amount in the tumor.

### BRIEF DESCRIPTION OF THE DRAWINGS

FIG. 1 shows a rate of tumor oxidative metabolism (OXm) plotted against radiation dose in patients with complete (CR) and partial (PR) remission. (*) P<0.05, compared to baseline.

FIG. 2 shows a mean tumor relative perfusion (rF) plotted against an accumulated dose in patients with complete (CR) and partial (PR) remission. No significant different was found between the two groups.

### DETAILED DESCRIPTION OF THE INVENTION

The present invention sets forth a link between alterations of the intermediary metabolism in cancer to treatment outcome. Specifically, tumor oxidative metabolism and nutritive perfusion are measured in vivo using ACE-PET. The present invention further relates to examining patients with head and neck cancer by investigating optimal PET tracer uptake revealed through Positron Emission Tomography (PET) that has more optimal staging than computer tomography (CT), Magnetic Resonance Imaging tomography (MRI) and FDG-PET.

PET imaging is a tomographic nuclear imaging technique that uses radioactive tracer molecules that emit positrons. When a positron meets an electron, they both are annihilated and the result is a release of energy in the form of gamma rays, which are detected by the PET scanner. By employing natural substances that are used by the body as tracer molecules, PET does not only provide information about structures in the body but also information about the physiological function of the body or certain areas therein. Furthermore, the choice of a tracer molecule depends on what is being scanned. Generally, a tracer is chosen that will accumulate in the area of interest, or be selectively taken up by a certain type of tissue, e.g. cancer cells. Scanning consists of either a dynamic series or a static image obtained after an interval during which the radioactive tracer molecule enters the biochemical process of interest. The scanner detects the spatial and temporal distribution of the tracer molecule. PET also is a quantitative imaging method allowing the measurement of regional concentrations of the radioactive tracer molecule. Commonly used radionuclides in PET tracers are ¹¹C, ¹⁸F, ¹⁵O ¹³N or ⁷⁶Br.

Furthermore, tracers labeled with short-lived positron emitting radionuclides (e.g. ¹¹C, t_{1/2}=20.3 min) are frequently used in various non-invasive in vivo studies in combination with PET. Because of the radioactivity, the short half-lives and the submicromolar amounts of the labeled substances, extraordinary synthetic procedures are required for the production of these tracers. An important part of the elaboration of these procedures is the development and handling of new ¹¹C- and ¹⁸F-labelled precursors. This is important not only for labeling new types of compounds, but also for increasing the possibility of labeling a given compound in different positions.

When compounds are labeled with ¹¹C, it is usually important to maximize specific radioactivity. In order to achieve this, the isotopic dilution and the synthesis time must be minimized Isotopic dilution from atmospheric carbon dioxide may be substantial when [¹¹C]carbon dioxide is used in a labeling reaction. Due to the low reactivity and atmospheric concentration of carbon monoxide (0.1 ppm vs. 3.4×10⁴ ppm for CO₂), this problem is reduced with reactions using [¹¹C]carbon monoxide.

In the current invention, ACE and ¹⁸F-acetate are developed as optimal PET tracers for not only diagnosing head and neck cancer in subjects but also to identify a subgroup of cancer patients in need of more advance treatments. There are several advantages in using PET technique and optimal PET tracers in the diagnosis of head and neck cancer. One advantage is that the methods of the instant invention provide optimal staging of this cancer is not reached in all patients using CT, MRI or FDG-PET. Another advantage is that the methods of the instant invention provide more advanced molecular imaging probes to allow the RT approach to be personalized thus opening doors for novel treatment opportunities, such as Intensity Modulated Radiation Treatment. Thirdly, in the cases where salivatory glands are non-functioning, the methods of the instant invention allows RT dose planning which does not need to avoid the glands and a higher radiation does could be given to the toumour without increased side effects.

After obtaining ACE and ¹⁸F-acetate, using an automated system such as FASTLAB® or TRACERLAB®, high performance liquid chromatography (HPLC) is used to verify the structure of the analogues. A further tool was used to verify the structure of the analogues wherein a calculation study was conducted to look into the physical properties and 3D images of various analogues. The calculation study can be conducted using a computer-aided molecular design modeling tool also known as CACHE®. CACHE® enables one to draw and model molecules as well as perform calculations on a molecule to discover molecular properties and energy values. The calculations are performed by computational applications, which apply equations from classical mechanics and quantum mechanics to a molecule.

Furthermore, in locally advanced head and neck cancer, a five-year progression-free survival is only 47% with combined radiotherapy, chemotherapy and surgery. Treatment failure is related to multiple factors, some of which are well characterized experimentally, but progress in terms of improving outcomes is slow. Tumor hypoxia is an important factor determining treatment response, as poor tumor oxygenation leads to radioresistance and local failure. Intracellular oxygen is needed to fixate DNA damage induced by radiation, but is also necessary for the tumor to maintain oxidative phosphorylation. Lack of oxygen resulting from insufficient perfusion would force the tumor cells to switch from respiration towards anaerobic glycolysis for survival. However, it is well known since the days of Warburg (Warburg O., On respiratory impairment in cancer cells. Science 1956; 124: 269-270) that many cancers share a common glycolytic phenotype, even in the presence of oxygen. Warburg attributed this phenomenon to a deranged mitochondrial function, causing impaired oxidative phosphorylation and disease progression. Recent in vitro studies along this line seem to confirm Warburg's notion. Tumor cells with deficient oxidative metabolic capacity represent a more malignant phenotype and oxidative metabolism may be a key factor in controlling cancer growth. Increased tumor glycolysis is detectable in vivo by [¹⁸F]-fluorodeoxyglucose (FDG)-PET and quantification of tumor FDG uptake using PET appears to carry prognostic information. Still, glucose uptake appears unrelated to the distribution of hypoxia. These findings imply that imaging of tumor oxidative metabolism and perfusion in vivo might provide insights into these mechanisms and ultimately predict tumor response.

Acetate has a pivotal role in the intermediary metabolism of all organisms and 1-[¹¹C]-Acetate (ACE) was developed and validated as a PET tracer of myocardial oxidative metabolism ten years ago. Exogenous ACE is vividly extracted into most tissues and the extraction rate approaches the rate of blood flow in, for instance, the myocard (17). Inside the cell, ACE is converted into [¹¹C]-acetyl-CoA and effectively trapped. In mitochondriae, terminal oxidation of [¹¹C]-Acetyl-CoA thru the tricarboxylic acid (TCA) cycle results in the formation of [¹¹C]-CO₂ and clearance of radioactivity from tissue by diffusion back into the circulation. More recently, ACE-PET has been used clinically for localizing various cancers that are not FDG-avid (Oyama N, Akino H, Kanamaru H, Suzuki Y, Muramoto S, Yonekura Y, et al. 11C-acetate PET imaging of prostate cancer. J Nucl Med 2002; 43: 181-186).

This aspect of imaging utilizes the fact that acetyl units not consumed in oxidation are used anabolically for proliferative support. Serial dynamic ACE PET scanning in patients treated with radiotherapy allows one to evaluate the role of tumor perfusion and mitochondrial function towards outcome in vivo.

Below a detailed description is given of non-invasive methods for assessing tumor perfusion and oxidative metabolism for in vivo imaging uses of PET tracers that are suitable in radiation therapy (RT) in head and neck cancer and evaluation of salivary gland function. A pharmaceutical comprising the compound and a kit for the preparation of the pharmaceutical are also provided.

In one embodiment of the invention, a non-invasive method for assessment of tumor perfusion and oxidative metabolism, comprising in vivo administration of a PET tracer in a subject with head and neck cancer is disclosed wherein the PET tracer may be ACE or ¹⁸F-acetate.

Another embodiment of the present invention is a non-invasive method for assessment of tumor perfusion and oxidative metabolism in a subject with head and neck cancer, comprising administration of a pharmaceutical composition of a PET tracer. Still a further embodiment of the current invention discloses a pharmaceutical composition comprising a PET tracer, together with a biocompatible carrier in a form suitable for mammalian administration.

Yet in another embodiment of the invention, a non-invasive method for assessment of tumor perfusion and oxidative metabolism comprising a personalized RT treatment for head and neck cancer in a subject comprising administering a pharmaceutical composition of a compound of a PET tracer, tracing tumor delineation and giving personalized radiation dose amount in the tumor is disclosed.

Still in a further embodiment of the present invention, the pharmaceutical composition comprising of the PET tracer, together with a biocompatible carrier in a form suitable for mammalian administration is claimed.

Yet another embodiment of the invention shows a kit comprising the PET tracer, or a salt or solvate thereof, wherein said kit is suitable for the preparation of a pharmaceutical composition thereof.

The kits comprise a suitable precursor of the second embodiment, preferably in sterile non-pyrogenic form, so that reaction with a sterile source of an imaging moiety gives the desired pharmaceutical with the minimum number of manipulations. Such considerations are particularly important for radiopharmaceuticals, in particular where the radioisotope has a relatively short half-life, and for ease of handling and hence reduced radiation dose for the radiopharmacist. Hence, the reaction medium for reconstitution of such kits is preferably a “biocompatible carrier” as defined above, and is most preferably aqueous.

A suitable kit container comprises a sealed container which permits maintenance of sterile integrity and/or radioactive safety, plus optionally an inert headspace gas (e.g. nitrogen or argon), whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. The “biocompatible cation” and preferred embodiments thereof are as described above.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the radioactive imaging product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

The term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The “biocompatible carrier” is a fluid, especially a liquid, in which the compound is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

Furthermore, the pharmaceutical compositions are suitably supplied in either a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. For radiopharmaceutical compositions, the pre-filled syringe may optionally be provided with a syringe shield to protect the operator from radioactive dose. Suitable such radiopharmaceutical syringe shields are known in the art and preferably comprise either lead or tungsten.

The radiopharmaceuticals may be administered to patients for PET imaging in amounts sufficient to yield the desired signal, typical radionuclide dosages of 0.01 to 100 mCi, preferably 0.1 to 50 mCi will normally be sufficient per 70 kg bodyweight.

Yet in another embodiment of the invention, a method for personalized RT treatment for head and neck cancer in a subject is claimed that comprises administering a pharmaceutical composition comprising a compound of a PET tracer, tracing tumor delineation and giving personalized radiation dose amount in the tumor.

Using standard RT approaches, the radiation dose deposited in the tumor is the same for all patients. Novel treatment opportunities, such as Intensity Modulated Radiation Treatment, will require more advanced molecular imaging probes to allow the RT approach to be personalized. One clinical problem is related to the tumor delineation and the differentiation of dose within the tumour. The tumour volumes derived from ACE and ¹⁸F-acetate PET images are significantly larger than volumes from FDG-PET, which demonstrates that radiolabelled acetate provide better tumour delineation for RT than existing methods.

The present invention also provides a non-invasive method for assessment of tumor perfusion and oxidative metabolism comprising a personalized RT treatment for head and neck cancer in a subject comprising administering a pharmaceutical composition of a compound of a PET tracer, evaluating salivary gland function, and giving personalized radiation dose amount in the tumor.

There is also a growing need to reduce RT dose to the normal tissues in order to avoid negative side effects, specifically salivatory glands of the head. In some cases, the salivatory glands are non-functioning and if these cases can be detected as part of routine scan, RT dose planning does not need to avoid the glands and a higher dose could be given to the tumour without increased side effects. ACE and ¹⁸F-acetate PET are valuable for the evaluation of salivary gland function. Incorporating this information into the dose planning algorithm increases the curative outcome of RT in head and neck cancer.

### EXAMPLES

The invention is further described in the following examples which are in no way intended to limit the scope of the invention.

### Experimental Studies

### Patients

The results of the study described below in nine patients with histologically confirmed squamous cell carcinoma of the head and neck were included into the study. All patients were untreated prior to this study and were candidates for radiotherapy. The clinical characteristics including the stage and the location of the primary tumors are shown in Table 1. Staging of the tumors was performed by CT or MRI, histopathology and clinical examination. All participating patients provided informed consent. The study of the nine patients indicate that ACE-PET scanning in subjects treated with radiotherapy would allow one to evaluate the role of tumor perfusion and mitochondrial function towards the outcome in vivo. Increased acetate uptake is a prominent feature of the primary tumors and lymph node metastases of head and neck squamous cell carcinomas were included in this study. ACE-PET provided diagnostic images of good quality and might be a more sensitive tool for staging of head and neck tumors than FDG-PET in a subset of cancer patients. The use of ACE-PET for tumor volume delineation resulted in 51% larger volumes than FDG-PET.

The clinical characteristics including the stage and the location of the primary tumors are shown in Table 1. Conventional staging of the tumors was performed by CT (n=9), MRI (n=1), histopathology and clinical examination. Histological confirmation was obtained by guided biopsies in all the primary tumors and most metastatic sites. The metastases not verified with biopsies (n=5) were deemed malignant based on the combination of all the available information and included a three month follow up. All patients participating in the study provided informed consent. The study was accepted by the ethical committee of the participating hospital.

### PET Imaging

Twenty nine dynamic ACE PET scans were performed in the nine patients. Five patients were scanned with a dedicated PET device (Siemens ECAT HR⁺, Knoxyille, Tenn., USA) and PET images were coregistered to dose-planning CT images for anatomical localization. Four patients were scanned with a hybrid PET-CT device (GE Discovery ST, Milwaukee, Wis., USA). ACE PET was studied in all patients within 7 days before the start of radiotherapy (baseline). Due to logistic problems not all patients could be scanned at all subsequent time points. Five patients were scanned after a mean dose of 15 Gy (dose range 9.6-20Gy), 7 patients after a mean dose of 30 Gy (range 24-37Gy) and 8 patients after a mean dose of 55 Gy (range 42-68Gy).

In a subset of ACE scan sessions (n=23) an image-derived arterial input function for absolute quantification of tumor perfusion was acquired by dynamic imaging of the heart immediately after injection of a 0.5 MBq/kg body weight ACE bolus.

Ten minutes after the heart scan, the head and neck region was imaged immediately after an intravenous bolus injection of 10 MBq/kg body weight ACE. The scan time was 32 minutes with time frames 12×5 seconds (s), 6×10 s, 4×30 s, 4×60 s, 2×120 s and 4×300 s.

FDG-PET was performed at baseline using a standard clinical whole-body protocol, in which the head and neck area was scanned one hour after intravenous injection of 5 MBq/kg body weight FDG. Baseline ACE and FDG scans were performed on the same or adjacent days.

### Acetate PET Imaging

Six patients were studied with dedicated PET and four patients were investigated with PET/CT. A 32 minutes dynamic emission scan was performed immediately after intravenous injection of 10 MBq/kg body weight ACE. The scan time was 12×5 s, 6×10 s, 4×30 s, 4×60 s, 2×120 s and 4×300 s. Frame 30 (17-22 minutes after injection) generally provided the best image quality with highest tumor to background ratio and was therefore chosen for subsequent data analysis.

### FDG PET Imaging

Whole-body scanning was performed one hour after intravenous injection of 5 MBq/kg body weight FDG. Six patients were examined by PET/CT and four patients were studied by PET alone. The patients were instructed to remain recumbent and avoid voicing and other uses of neck muscles during the uptake period.

### Data Analysis

PET images were co-registered with the CT or MRI images in all patients by a normalized mutual information procedure supported by manual correction using Hermes MULTIMODALITY™ software (Nuclear Diagnostics, Stockholm, Sweden). FDG-PET and ACE-PET images were analyzed both qualitatively and quantitatively, using Hermes VOLUME DISPLAY™ version V2β. In qualitative analysis, PET images were interpreted visually by two nuclear medicine physicians and any disagreement was resolved by consensus. The tumor uptake of FDG and ACE were graded into negligible, mild, moderate and intensive compared to the contra-lateral or surrounding tissues. An abnormal uptake equal to or exceeding mild was considered positive. In quantitative analysis, the mean standardized uptake value (SUV) and tumor volumes delineated by ACE and FDG-PET were evaluated. SUV was calculated as mean radioactivity concentration in the volumes (Bq/cc) divided by injected dose (Bq) per kilogram body weight. For lesions with negligible uptake, similar tumor volumes were drawn manually by visual correlated fusion images.

Each tumor volume in FDG-PET and ACE-PET was delineated automatically by tracing an isoactivity pixel value set to 50% threshold of the maximum radioactivity corrected for background. The background was measured from a separately drawn region of interest (ROI) adjacent but at safe distance from the tumor. The isoactivity pixel value of each volume was calculated as:

Isoactivity pixel value=(MPVₜᵤₘₒᵣ+APV_{background})×50%

MPV is the maximum pixel value and APV is the average pixel value of the background ROI. This approach takes into account the variable background activity, effectively cancels the effect of varying background uptake on tumor volume measurements and was found to be highly reproducible. In those cases where the tumor location was near to the salivary glands with normally high physiological uptake of ACE, the tumor volumes were adjusted manually based on the combined information of CT and PET. Only one primary tumor volume and five metastases needed manual adjustments due to this reason.

### Statistical Analysis

The relationship between FDG SUV and ACE SUV was determined by Pearson's correlation coefficient. ANOVA test was used to compare the tracer uptake with histological cell differentiation. The differences between the FDG and ACE SUVs and volumes were analyzed by nonparametric Wilcoxon signed rank test. Volumes of metastases were presented by median±interquartile, since it did not show a normal distribution. A p value <0.05 was considered statistically significant. Calculations were performed by SPSS version 11.5.

### Tumor Clearance Rate

Primary tumors were clearly visualized in all scans. A time-activity curve (TAC) of the primary tumor was obtained from a region of interest (ROI) delineating the highest uptake in all ACE scans. TACs were analyzed by fitting an exponential curve to the data collected between 4 and 32 min Tumor oxidative metabolism was derived from the below equation.

Y=Ae^{-OXm*t}

where Y is the tumor radioactivity (Bq/cc), A is a constant, t is time (min), and OXm is the clearance rate of [¹¹C] in min⁻¹. The average R² of the fit was 0.93.

### Tumor Perfusion

The arterial blood input function was derived by placing a small ROI in the left ventricular cavity of the heart scan to obtain a TAC of the first pass of the bolus. Arterial blood activities were integrated by the area-under-the-curve of the first bolus passage through the chamber and normalized to the injected dose of the subsequent tumor scan.

The peak activity of the primary tumor deposited at the end of the first bolus pass was assessed. The absolute extraction rate (in mL/min/mL tissue) was calculated by dividing the first-pass peak tumor activity by the arterial blood integral, assuming that systemic circulation was unaltered between the heart and tumor scan. As the arterial input was not obtained in all sessions, a relative perfusion index (rF) was quantified by the ratio of initial peak retention in the tumor to that of the cerebellum. The cerebellum was chosen as a reference because this region was excluded from radiotherapy and had a highly stable extraction rate (0.08±0.03 mL/min/mL) in all patients with minimal variation between scan sessions. Absolute quantification of tumor perfusion in 23 scans yielded a mean of 0.47±0.01 mL/min/mL The mean of simultaneous rF measurements was 5.87±0.39 and the two methods were linearly correlated (r=0.57, p=0.005).

### Tumor Glucose Uptake

Tumor glucose uptake (Tglu) was measured from the FDG data as standard uptake values (SUV), determined by the radioactivity concentration of the tumor ROI, divided by injected activity per gram body weight.

### Radiotherapy and Tumor Response

External beam radiotherapy was delivered with a standardized technique to the primary tumor and lymph node metastases using 3D treatment planning Total dose was 68 Gy, generally with 2 Gy per fraction and 5 fractions per week.

The outcome was evaluated by clinical examination, panendoscopy, and CT or MRI scanning 6-8 weeks after completed radiotherapy. Tumor response was recorded as complete response (CR), partial response (PR), stable disease and progressive disease according to standard criteria (22). One patient was operated after radiotherapy due to aggressive tumor growth. Patients were followed up regularly until the submission of this paper or death. The median follow up time was 26 months (range 1.5-45 months). Six patients were considered CR (Table 2), of which 5 were alive at the time of submitting. Three patients showed PR and all died during follow-up. There was no difference of the prescribed radiation dose between the CR and PR patient groups.

### Results:

### Tumor Oxidative Metabolism

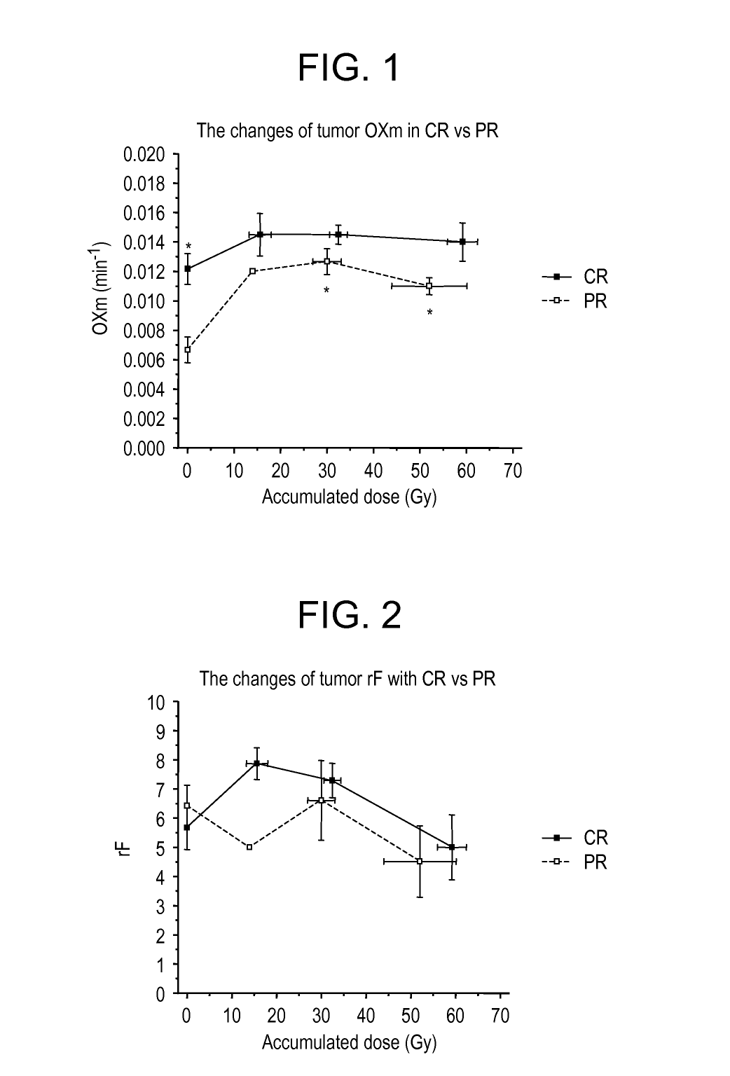

Tumor OXm values are presented in Table 2 and FIG. 1. Before radiotherapy, the mean OXm of CR was almost double to that of PR (p=0.02) with no overlap between groups. OXm of CR did not change significantly during radiotherapy. In contrast, the OXm of PR was significantly increased at 30 Gy (p=0.002) and 55 Gy (p=0.008), compared to baseline. Only one patient was scanned at 15 Gy in PR and therefore not included in ANOVA analysis. OXm was not significantly different between CR and PR at 30Gy or 55 Gy.

### Tumor Perfusion

Table 3 and FIG. 2 describe the primary tumor rF of CR versus PR. In the CR group, tumor rF tended to increase from baseline to 15 Gy (p=0.06), was relatively stable at 30 Gy and then decreased at 55 Gy (p=0.03, compared with the rF at 15 Gy). No significant changes of rF was observed in PR (p=0.41). No difference of rF between CR and PR at same dosages was found.

### Tumor Glucose Uptake

Increased FDG uptake was seen in all primary tumors (Table 1) and Tglu was 10.9±2.4 SUV. Tglu was significantly higher in PR than CR (p=0.04).

### Correlations

A positive correlation of overall OXm and rF was found in the CR (r=0.69, p=0.001) and this correlation was almost perfect at baseline (r=0.93, p=0.008). OXm and rF were not correlated in PR. Baseline Tglu tended to correlate inversely with OXm (r=−0.57, p=0.11), but was not significantly correlated with rF.

### CONCLUSION

This study probed the intermediary metabolism of human cancers towards the response to radiotherapy using non-invasive molecular imaging methodology. Terminal clearance rate of carbon units from tumor tissue was used as an index of tumor oxidative metabolism and was significantly lower in patients with poor outcome Impairment of oxidative metabolism was associated with increased glycolysis in spite of intense perfusion. Tumor perfusion was substantial in all cancers, but was coupled to the oxidative metabolic rate only in cancers with favorable outcome.

The data disclosed herein shows in vivo the existence of a bioenergetic switch from oxidative metabolism towards aerobic glycolysis associated with resistance to radiotherapy in head and neck cancer. Baseline assessment of OXm predicted treatment outcome. The lowest OXm rates were recorded in tumors with partial response and all PR patients died within 26 months after radiotherapy. Noteworthy, patient No 3 was staged as T2NOMO with a highly differentiated tumor, but died within 6 months due to aggressive tumor growth and metastasis. This patient had the lowest OXm and the highest TGlu of all patients, indicating that the bioenergetic shift impacts outcome and is not immediately apparent with standard diagnostic evaluation.

There are probably several different mechanisms by which cancer cells develop a bioenergetically inferior glycolytic phenotype. Previous work has pointed out that these changes are associated with mitochondrial DNA mutations, hypoxia, and altered regulation of enzymes in both bioenergetic pathways as well as accelerated proliferation. Most likely, this phenotype facilitates survival either by minimizing oxygen dependence or by downregulating the pro-apoptotic role of mitochondriae. Our data does not allow causal conclusions, but extends the previous in vitro findings regarding the relevance of tumor bioenergetics into the clinical situation. From a therapeutical point of view, a recent study indicated that forcing tumor cells from glycolysis into mitochondrial oxidative metabolism inhibited cancer growth and postulated that tumor invasiveness might be inversely linked to respiration. Quantitative metabolic imaging might be crucial for stratifying patients in trials along this line.

OXm increased during radiotherapy in PR tumors, raising the possibility that mitochondrial dysfunction in radioresistant cancers is reversible. As perfusion was relatively unchanged by radiation in this group, passive reoxygenation from decompression and reperfusion might not explain the OXm increase alone. Cancer cells grown in 4% O₂ increase their oxygen consumption and die sooner than normoxically grown cells when treated with low-dose radiation, suggesting that the role of hypoxia as an outcome predictor has not been fully elucidated. Altered substrate availability alone may change mitochondrial function. Further, cancer cells with reduced oxidative metabolism increase their mitochondrial mass during radiotherapy, which could also explain the finding. Still, mitochondrial responses to radiotherapy in vivo are poorly understood and more integrative approaches are probably needed for improved translational research in this area.

ACE PET provided quantitative estimates of nutritive perfusion and oxidative metabolism. Metabolism was assessed by calculating OXm, the rate of [¹¹C] clearance from tissue, by a simple fitting procedure. OXm and ACE-PET is the golden standard for non-invasive measurements of regional oxygen consumption in myocardial tissue and was recently also validated in a renal animal model. Myocardial O₂ consumption in resting normal volunteers is 3-4 micromole/min/gram, associated with OXm values of 0.05-0.07 min⁻¹ Correspondingly, OXm values in cancers were substantially lower than that of myocard. It is not known whether OXm from different tissues are directly comparable in absolute terms. The values obtained appear meaningful in the context of this material and further validation is warranted.

Kinetic estimation of perfusion using PET requires a blood input function from true arterial samples or from a substantial blood compartment in the image. Arterial sampling was deemed too invasive in this study and intravascular activity in neck vessels can not be accurately measured by PET, due to partial volume effects. Therefore we assessed arterial ACE activity from near simultaneous left ventricular blood pool imaging, a standard method in quantitative cardiac PET. Absolute ACE extraction rate averaged 0.47 mL/min/mL, approaching the perfusion rate recorded in healthy myocardium at rest using the same technique and that of previous data in human cancers using other methods. As this approach added to the complexity of the study, cardiac scans were not obtained at all time points. Substituting blood activity with a cerebellar reference successfully accomplished a simple index of nutritive tumor perfusion.

It is an axiom in normal physiology that regional perfusion is dictated by tissue demand. OXm and rF were highly correlated in CR patients. This is a novel finding indicating both that this axiom is valid in radiosensitive cancers and that these tumors relied predominantly on respiration for energy formation even during radiotherapy. Perfusion of CR tumors tended to increase at 15 Gy and then decreased at 55 Gy. This finding fits well with the concept that cell death early during successful radiotherapy causes tumor decompression, leading to reperfusion. At the end of therapy, when most tumor cells were killed, total metabolic demand was reduced and less blood flow was needed. Reports on tumor perfusion during treatment in vivo are scarce and somewhat contradictory. Perfusion, as measured in the present study, was not directly related to outcome.

Dynamic ACE PET allowed simultaneous and non-invasive evaluation of tumor oxidative metabolism and perfusion in head and neck cancer patients with a single tracer injection, a short scanning protocol and simple evaluation techniques. Visualization of tumor masses was excellent. The use of the method is limited to PET facilities with on-site cyclotrons, which is a drawback. The limited number of patients might have affected the interpretation and confirming as well as validating studies are needed.

Accordingly, the present invention presents a new non-invasive method for simultaneous assessment of tumor perfusion and oxidative metabolism in patients using dynamic ACE-PET. This method can be used to document a metabolic abnormality, predictive of poor response to radiotherapy. Restoration of tumor oxidative metabolism is a potential target for improvement in cancer therapy.

TABLE 1
Patient clinical characteristics: M = male, F =
female, diff = cell differentiation

| Patient | | Age | | | | Histology |
|---|---|---|---|---|---|---|
| No | Sex | (year) | Stage | Location | Tglu | diff |
| 1 | M | 77 | T4N2cM0 | Larynx | 13.8 | Low |
| 2 | F | 57 | T2N0M0 | Nose | 3.9 | Moderate |
| 3 | M | 59 | T2N0M0 | Nose | 26.1 | High |
| 4 | M | 53 | T3N0M0 | Nose/sinus | 4.6 | Low |
| 5 | F | 67 | T4N3M1 | Tonsilla | 13.4 | Low |
| 6 | M | 59 | T3N1M0 | Tonsilla | 8.5 | Low |
| 7 | M | 47 | T4N3M0 | Epipharynx | 6.8 | Low |
| 8 | M | 64 | T2N2aM0 | Tonsilla | 4.9 | Low |
| 9 | M | 18 | T3N3M0 | Epipharynx | 16.3 | Low |

TABLE 2
The tumor oxidative metabolic rate (OXm, unit
min⁻¹) serially measured during radiotherapy.

| TR | Patient No | Baseline | 15 Gy | 30 Gy | 55 Gy |
|---|---|---|---|---|---|
| CR | 1 | 0.0115 | ND | ND | 0.0127 |
| CR | 2 | 0.0121 | 0.0178 | 0.0145 | 0.0181 |
| CR | 4 | 0.0097 | 0.0109 | 0.0130 | 0.0143 |
| CR | 6 | 0.0124 | ND | 0.0136 | 0.0097 |
| CR | 7 | 0.0173 | 0.0146 | ND | ND |
| CR | 8 | 0.0099 | 0.0135 | 0.0164 | 0.0153 |
| Mean ± SE | | 0.0122 ± | 0.0142 ± | 0.0144 ± | 0.0140 ± |
| | | 0.0011 | 0.0015 | 0.0008 | 0.0014 |
| N | | 6 | 4 | 4 | 5 |
| PR | 3 | 0.0051 | ND | 0.0128 | 0.0120 |
| PR | 5 | 0.0078 | ND | 0.0109 | 0.0104 |
| PR | 9 | 0.0065 | 0.0116 | 0.0140 | 0.0105 |
| Mean ± SE | | 0.0065 ± | ND | 0.0126 ± | 0.0110 ± |
| | | 0.0008 | | 0.0009 | 0.0005 |
| N | | 3 | 1 | 3 | 3 |

TR = tumor response; CR = complete response; PR = partial response; rF = relative perfusion; N = number of patients undergoing ACE PET per group and dosage. ND = no data available.

TABLE 3
describes the primary tumor rF of CR versus PR.

| | Patient No | Baseline | 15 Gy | 30 Gy | 55 Gy |
|---|---|---|---|---|---|
| CR | 1 | 6.26 | ND | ND | 2.81 |
| CR | 2 | 4.90 | 8.22 | 8.06 | 7.76 |
| CR | 4 | 3.96 | 7.07 | 6.54 | ND |
| CR | 6 | 5.91 | ND | 6.05 | 3.64 |
| CR | 7 | 8.93 | 9.25 | ND | ND |
| CR | 8 | 4.10 | 6.93 | 8.51 | 5.78 |
| Mean ± S E | | 5.68 ± | 7.87 ± | 7.29 ± | 5.00 ± |
| | | 0.75 | 0.54 | 0.59 | 1.11 |
| N | | 6 | 4 | 4 | 5 |
| PR | 3 | 7.63 | ND | 7.39 | 5.00 |
| PR | 5 | 6.47 | ND | 8.48 | 6.34 |
| PR | 9 | 5.17 | 5.00 | 3.95 | 2.20 |
| Mean ± SE | | 6.42 ± | ND | 6.61 ± | 4.51 ± |
| | | 0.71 | | 1.37 | 1.22 |
| N | | 3 | 1 | 3 | 3 |

In the CR group, tumor rF tended to increase from baseline to 15 Gy (p = 0.06), was relatively stable at 30 Gy and then decreased at 55 Gy (p = 0.03, compared with the rF at 15 Gy). No significant changes of rF was observed in PR (p = 0.41). No difference of rF between CR and PR at same dosages was found.

### SPECIFIC EMBODIMENTS, CITATION OF REFERENCES

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A non-invasive method for assessment of tumor perfusion and oxidative metabolism, comprising in vivo administration of a PET tracer in a subject with head and neck cancer.

2. A method of claim 1, wherein the PET tracer is ACE.

3. A method of claim 1, wherein the PET tracer is ¹⁸F-acetate.

4. A non-invasive method for assessment of tumor perfusion and oxidative metabolism in a subject with head and neck cancer, comprising administration of a pharmaceutical composition of a PET tracer.

5. A method of claim 4, wherein the pharmaceutical composition comprises the PET tracer, together with a biocompatible carrier in a form suitable for mammalian administration.

6. A kit comprising the PET tracer, or a salt or solvate thereof, wherein said kit is suitable for the preparation of a pharmaceutical composition of claim 4.

7. A non-invasive method for assessment of tumor perfusion and oxidative metabolism comprising a personalized RT treatment for head and neck cancer in a subject comprising administering a pharmaceutical composition of a compound of a PET tracer, tracing tumor delineation and giving personalized radiation dose amount in the tumor.

8. A non-invasive method for assessment of tumor perfusion and oxidative metabolism comprising a personalized RT treatment for head and neck cancer in a subject comprising administering a pharmaceutical composition of a compound of a PET tracer, evaluating salivary gland function, and giving personalized radiation dose amount in the tumor.
